(19) United States
(12) Patent Application Publication
(10) Pub. No.: US 2010/0235981 A1
(43) Pub. Date: Sep. 23, 2010

(54) CONTAINER UNIT FOR STORAGE AND DISINFECTION

(76) Inventor: Vivi Aakjaer Jensen, Skive (DK)

Correspondence Address: JAMES C. WRAY, 1493 CHAIN BRIDGE ROAD, SUITE 300, MCLEAN, VA, 22101, US

(21) Appl. No.: 12/451,928

(22) PCT Filed: Jun. 4, 2008
(86) PCT No.: PCT/DK2008/000207

§ 371 Date: May 25, 2010

(30) Foreign Application Priority Data
Jun. 5, 2007 (DK) PA 2007 00813

(51) Int. Cl. E03C 1/18; B65D 25/02; A61L 2/00

(52) U.S. Cl. 4/628; 220/735; 422/300

(57) ABSTRACT

Container unit for storage and disinfection of various items, such as dishcloths, sponges, brushes and the like, where the unit comprises an outer housing, and an inner moveable rack, characterised in that the inner moveable rack comprises a reservoir, and that said rack and reservoir are connected to the outer housing by means whereby the inner rack can move out from a position in the housing where the housing substantially encloses the rack and reservoir, to a position where the rack and at least part of the reservoir are accessible, and that means are provided either in the housing or the rack for disinfecting objects on the rack.

### FIELD OF THE INVENTION

The present invention relates to a container unit for storage and disinfection of various items and is also directed to a sink, in particular a kitchen sink in which a container according to the invention has been incorporated.

### BACKGROUND OF THE INVENTION

It is a general problem to be able to keep the hygiene standard high in environments where bacteria growth may be present in the vicinity of food or other places where high hygienic requirements are required.

For example in kitchens it is quite usual to use a dishcloth, sponge or the like to wipe off the table top surfaces, refrigerators, cooking surfaces etc., and it does not take a very long time for bacteria growth to explode in a hot and humid environment as is usually present in a kitchen, such that from being a means for cleaning, i.e. a dishcloth, it becomes a tool with which the bacteria may spread to other parts of the kitchen environment.

In order to maintain a high hygienic standard it would therefore be necessary to discard the dishcloth, sponges, brushes etc. each time they had been used in order for the bacteria growth not to explode and contaminate the environment further. Usually, and especially in domestic households this is not done, and there is therefore a latent risk that dishcloth, towels, sponges, brushes etc. may be the source of contamination or cross contamination between raw meat, vegetables, dishes etc.

### OBJECT OF THE INVENTION

It is therefore an object of the present invention to provide a solution whereby it becomes easier and more convenient to ensure that objects such as dishcloths, sponges etc. are maintained at a safe, hygienic standard at all times.

### DESCRIPTION OF THE INVENTION

The invention addresses this problem by providing a container unit for storage and disinfection of various items, such as dishcloths, sponges, brushes and the like, where the unit comprises an outer housing, and an inner moveable rack, characterised in that the inner moveable rack comprises a reservoir, and that said rack and reservoir are connected to the outer housing by means whereby the inner rack can move out from a position in the housing where the housing substantially encloses the rack and reservoir, to a position where the rack and at least part of the reservoir are accessible, and that means are provided either in the housing or the rack for disinfecting objects on the rack.

The movable rack may be extended outside the housing such that it becomes easy to place the objects such as dishcloth, brush, sponge or the like in the rack after which the rack is lowered inside the housing where the disinfection of the objects takes places.

Furthermore, by lowering the rack inside the housing of the container unit, the objects are hidden and thereby out of the way such that a more aesthetic (uncluttered) environment around the container unit is created.

The reservoir tends to accumulate liquids that may drop from the objects and furthermore in embodiments which will be described below where a liquid disinfectant is utilized the surplus disinfectant may also be collected by the reservoir. Advantageously, the reservoir is removably mounted in the rack such that it may be removed for emptying and cleaning at convenient intervals.

In a further advantageous embodiment the means for moving the rack relative to the housing is a gas loaded spring mechanism and a snap lock mechanism. In this manner it becomes possible simply by pushing on the top of the rack to release the snap lock mechanism whereby the loaded gas spring will push the rack upwards thereby exposing the rack from the housing making the objects placed on the rack accessible. Furthermore, by simply pushing the rack downwards until the snap lock mechanism engages, the rack will be safely held within the housing such that proper disinfection may take place and at the same time the objects placed on the rack are hidden inside the container.

In a further advantageous embodiment the means for moving the rack relative to the housing is a rack and pinion or spindle system such that by activating the mechanical system, for example by means of a motor, the rack will automatically extend from the housing making the object stored on the rack accessible.

This electro-mechanical construction is also a very safe and well-proven principle which may be used reliably to create the movements necessary in order to hide respectively expose the rack as already discussed above.

In a still further advantageous embodiment of the invention the container unit is furthermore on top of the rack provided with a closure member such that when the rack and reservoir is inside the housing, the closure member seals liquid tight against the outer housing. The provision of the liquid tight sealing against the outer housing is provided for a number of reasons. Firstly, it is important that when liquids are used for disinfecting the objects placed on the rack that this liquid is not accidentally spread to the environment immediately adjacent the container unit whereby it could come into contact with food placed in the vicinity. Furthermore, it is furthermore not desirable to have liquids from the environment entering the housing as this would fill up the reservoir and/or dilute the disinfection process or contaminate objects placed on the rack which have already been disinfected. The closure member is therefore an important feature for the container unit, especially in environments where possible contaminants are handled immediately adjacent the container unit. It is not necessary that the closure member also creates a vapour tight seal in that such a construction in for example a kitchen environment would be very hard to maintain in that typically vapour tight seals are rather fragile and need only to be polluted slightly before the vapour tightness is void. On the other hand, it is a relatively easy and well established technology to provide liquid tight seals between mechanical members of this type.

In a further advantageous embodiment the means for disinfection comprises one or more nozzles arranged either in the wall of the housing or integrated in the rack, where said nozzles are connected to a disinfectant reservoir via a pump, which pump may either be activated automatically when the rack is inside the housing, or is manually activated.

The nozzles will create a mist inside the container and around the objects placed on the rack such that an effective disinfection is created on the objects placed on the rack. The surplus disinfectant will be collected in the reservoir and may be disposed of in a suitable manner, whereby a very safe and reliable system of disinfection is created. The pump may be may be any suitable pump which is able to pump the disinfectant liquid from a storage container to the nozzles arranged on the inner wall of the housing or integrated in the rack.

By providing the options of either automatically activating the disinfection means when the rack is lowered into the housing or manually activating the disinfection means various advantages are achieved. By automatically activating the disinfection means it is ensure that each the time the rack is lowered inside the housing, the objects placed on the rack will be disinfected. On the other hand, if the objects on the rack are stored for a relatively long period of time, and the disinfection for one reason or the other is not 100% effective, a bacteria culture could propagate such that the effect of the disinfection cycle is relative small. For this purpose it may be advantageous to be able to instigate the disinfection manually such that for example before the rack is elevated from the housing a disinfection cycle is carried out it will be ensured that the objects on the rack when being exposed and made accessible due to the elevation of the rack that the objects are recently disinfected.

In a further advantageous embodiment the disinfection comprises one or more ultraviolet light sources, where the lights are activated either automatically or manually when the rack is inside the housing. In some environments it may be advantageous not to use liquid based disinfectants but use ultraviolet light depending on the bacteria and pollution in that particular environment. For these purposes this embodiment suggests that ultraviolet light sources arranged inside the housing or integral with the rack may be used in order to exterminate the bacteria which may be present on the objects. As was the case and with the same advantages as already mentioned above the disinfection means in the shape of ultraviolet light sources may be activated either automatically or manually as already discussed above.

The housing may in a further advantageous embodiment be provided with mounting means on the outside of the wall of the housing, where said means are adapted to engage a surface, such as a table top, kitchen sink or the like. The mounting means serves to fasten the container unit to a surface or in a surface such as for example a kitchen top, a flange on a sink or the like, i.e. in any convenient position such that the container unit may be arranged in any convenient position.

In yet a still further advantageous embodiment a separate push button device is provided, either separately from the container or integrated in the rack or closure member, where said push button device is connected to the disinfecting means and/or the means for moving the rack for activation of said means. A separate push button provides the advantage that a user does not need to touch the rack or container unit during operation of the device but may simply by operating a push button carry out the necessary actions, i.e. elevating or lowering the rack or initiating the disinfecting cycle.

The push button may be integrated in the closure member if a closure member is provided or may be a completely separate member mounted in the vicinity of the container unit according to the invention.

The invention also relates to a sink, in particular a kitchen sink, where said sink comprises a basin and an upper flange surrounding said basin where a container according to any of the embodiments mentioned above is integrated or mounted in said flange. Typically, the flange will extend at a substantially right angle to the walls of the basin where the flanges are suitable to be arrange flush or immediately above the top surface of for example a work top in a kitchen, lavatory, workshop or the like, and as such by arranging the container unit immediately adjacent the basin it is easy and convenient to place the objects such as dishcloths, wipes, brushes and the like after use.

Above the invention has been described with respect to a container unit and a rack where objects such as dishcloths, wipes, sponges, brushes and the like may be stored. For other purposes the container unit may have a size such that it is also possible to place other objects, such as for example soap dispensers, detergent bottles and the like on a shelf next to the reservoir, such that by lowering the rack into the container unit a clutter-free environment is provided around the sink.

When the container unit according to the invention are installed in for example bathrooms extra space may be provided on the rack for toothbrushes, toothpaste and other objects. The additional objects may be placed in a compartment inside the container which is not exposed to the disinfectant.

As should be evident from the description above, the container may be manufactured in any suitable material, such as for example plastics, metal, composites, glass, china and the like. It should, however, be emphasized that when used in connection with industrial applications such as kitchens, lavatories and the like, stainless steel is a preferred material in that stainless steel withstands rather harsh detergents which are often required during the thorough cleaning of such places.

It should also be obvious that the container unit according to the invention and as presented above may be provided as a separate unit which can be mounted in any table top surface provided with an adequate aperture it may also be integrated, i.e. made as an integral part of for example a kitchen sink such that no welds or the like will be present. The shape of the container unit may be chosen completely freely in that as long as the requirements to providing a rack construction which can be elevated relative to the housing, any cross sectional shape of the housing may be used such as for example circular, oval, rectangular, square and the like.

### DESCRIPTION OF THE DRAWING

The invention will now be explained with respect to the accompanying drawing wherein

FIGS. 1, 2 and 3 are perspective views of different embodiments;

FIGS. 4 and 5 are cross sections through an embodiment according to FIG. 3, and

FIGS. 6 and 7 are illustrations of various sink lay-outs incorporating the inventive container unit.

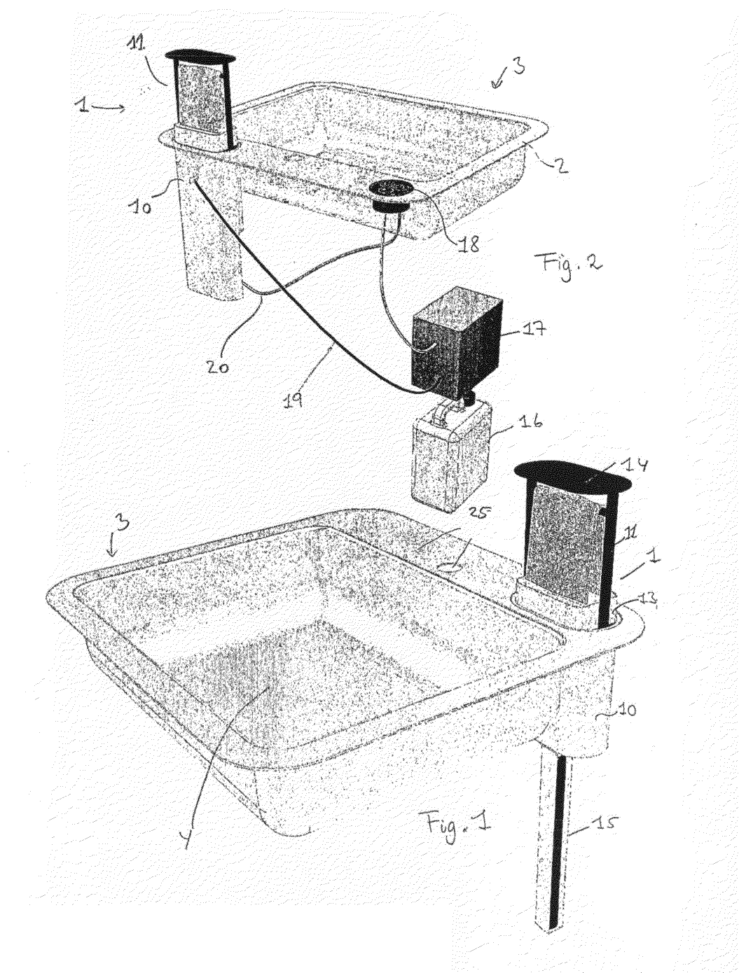

In FIG. 1 is illustrated a container unit according to the invention mounted in a flange on a sink. The container unit 1 is mounted in a flange 2 of a sink 3. Inside the flange 2 is provided a basin 4 and an aperture 5 is provided in which a water tap or the like may be arranged.

The container unit 1 comprises a housing 10, a movable rack 11, a closure member 12 and a removable reservoir 13. In this embodiment as is the case with all the embodiments which will be described for the specific embodiment of the invention, the rack 11 is designed to accommodate a dishcloth 14, but any suitable design of the rack 11 may be provided in order to accommodate various objects.

In the illustration in FIG. 1 the rack 11 is in its elevated position relative to the housing 10 such that the dishcloth 14 is accessible. The elevation is provided by means of a gas spring 15 mounted at the bottom of the housing 10 and connected to the bottom of the rack 11 such that by depressing the closure member 12 the gas stream will be compressed and a lock mechanism will engage the closure member or the rack as it comes into its closed position. By furthermore slightly pushing on the closed container unit, for example on the closure member 12, the lock mechanism will be released and the gas spring will propel the rack into the position illustrated with reference to FIG. 1.

In FIG. 3 is illustrated a further embodiment of the invention, where the rack is in its closed position.

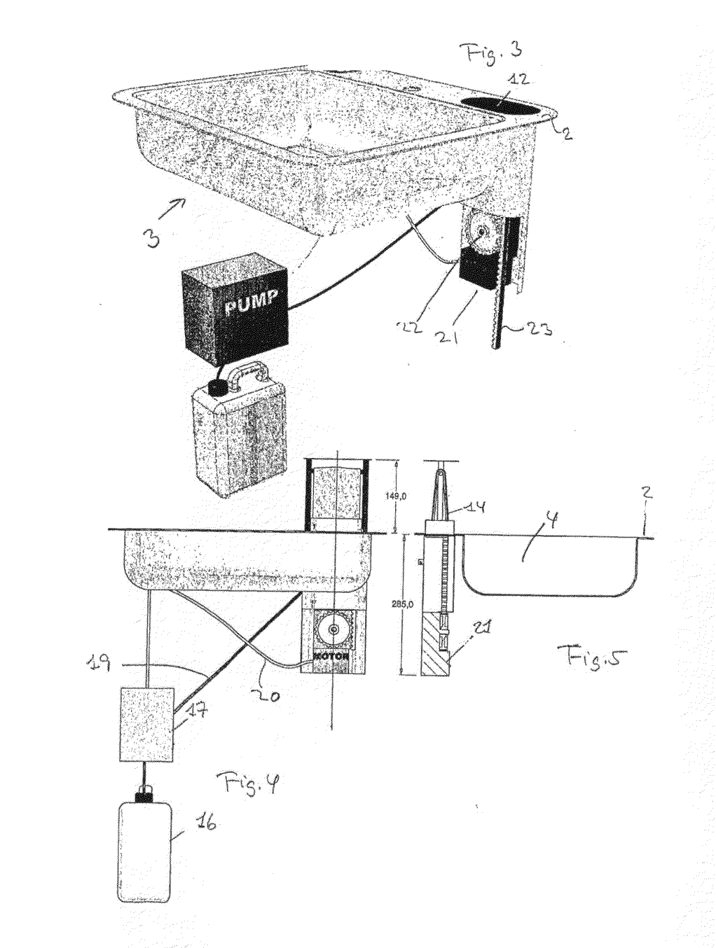

Turning to FIG. 2 an embodiment where the elevating mechanism comprises an electrical motor is illustrated which will be further explained with reference to FIGS. 3 and 4. The disinfection unit is visible and comprises a container 16 in which the disinfectant is stored. The container 16 is by means of a tube connected to a pump and dispensing unit 17 which when activated by a push button device 18 in this embodiment mounted in a flange 2 of the sink 3 pumps disinfectant through the pipe 19 through the housing 10 of the container unit 1.

In the rack's 11 elevated position as indicated in FIGS. 1 and 2 it is not possible to activate the pump 17. The push button device 18 furthermore comprises means for controlling the elevating mechanism which is activated by means of the wire 20. For clarity reasons the general power supply is not illustrated, but in most kitchens, lavatories and similar places, power outlets are readily available such that the electrical connections for the installation as illustrated in FIG. 2 is relatively easy to establish.

Turning to FIG. 3 a similar installation as described with respect to FIG. 2 is presented, but the closure member 12 is in this embodiment in a position where it is substantially flush with the flange 2 of the sink 3. The elevating mechanism for elevating the rack 11 in this embodiment comprises a motor 21 which drives a toothed wheel 22 which wheel is in engagement with a toothed rack 23 which toothed rack 23 is connected to the rack 11 such that by rotating the wheel 22 the rack 23 will move up and down thereby exposing the rack 11 or closing the container unit. In FIGS. 4 and 5 are illustrated cross sections at right angles to each other through the invention as illustrated in FIG. 3.

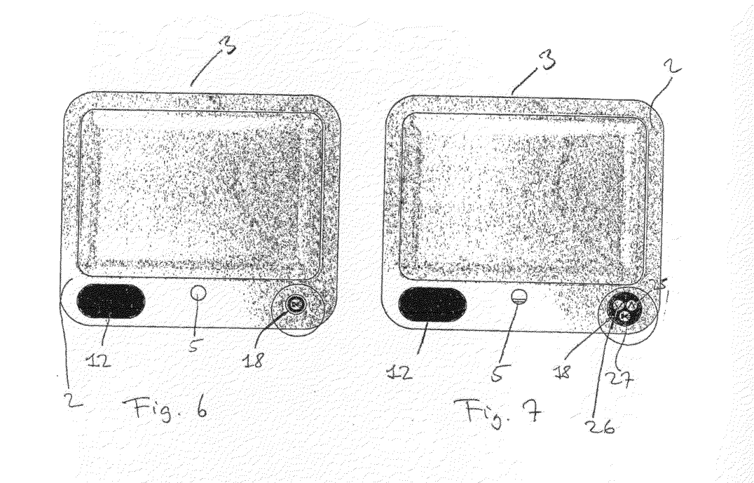

In the further FIGS. 6 and 7 a sink incorporating a container unit according to the invention is illustrated. The container unit is visible by its closure member 12 which is arranged in a flange portion 2 of the sink 3. In FIG. 6 the push button device 18 only has one function namely to activate the disinfection for example by activating the pump 17 described with reference to FIGS. 2 and 3.

In FIG. 7 the push button device 18 has additional functions. The push button device 18 in this embodiment is in the shape of a touch button where a button 25 for elevating the rack 11 and a button 26 for closing the container unit is provided in addition to the disinfection button 27.

The arrangement of the nozzles inside the housing will be carried out in a manner such that an effective mist generation inside the closed container unit will be provided in order to facilitate a thorough disinfection. The same is true for the embodiments of the invention where disinfection is carried out by ultraviolet light sources in which cases the pumps 17 and disinfection containers 16 are not necessary but solely electrical power is necessary connected to the housing in order to generate the ultraviolet light.

Above the invention has been described with reference to a specific embodiment in the shape of a kitchen sink, but it is contemplated within the scope of the invention that the container unit 1 may be used in connection with a number of other applications such as for example sinks and kitchens, workshops, canteens, hospitals, public pools, housing for the elderly, educational facilities, for example school kitchens, physics and chemistry instruction rooms, print shops etc. etc. Furthermore in health spas, public toilets, adjacent barbecues or on food trolleys and the like it is very simple to incorporate a container unit according to the present invention which will facilitate and improve hygienic standards for the particular application.

Although the invention has been described with reference to various embodiments the scope of protection of the invention shall only be limited by the appended claims.

1. Container unit for storage and disinfection of various items, such as dishcloths, sponges, brushes and the like, where the unit comprises an outer housing, and an inner moveable rack, characterised in that the inner moveable rack comprises a reservoir, and that said rack and reservoir are connected to the outer housing by means whereby the inner rack can move out from a position in the housing where the housing substantially encloses the rack and reservoir, to a position where the rack and at least part of the reservoir are accessible, and that means are provided either in the housing or the rack for disinfecting objects on the rack.

2. Container according to claim 1 wherein the means for moving the rack relative to the housing is a gas loaded spring mechanism and a snap lock mechanism.

3. Container according to claim 1 wherein the means for moving the rack relative to the housing is a rack and pinion or spindle system.

4. Container according to claim 1 wherein the means for moving the rack relative to the housing is an electrical motor connected by means of a flexible belt to one or more pulleys on the rack, such that by activating the motor the rack moves relative to the housing.

5. Container according to claim 1 wherein on top of the rack a closure member is arranged, such that when the rack and reservoir is inside the housing, the closure member seals liquid tight against the outer housing.

6. Container according to claim 1 wherein the means for disinfection comprises one or more nozzles arranged either in the wall of the housing or integrated in the rack, where said nozzles are connected to a disinfectant reservoir via a pump, which pump may either be activated automatically when the rack is inside the housing, or is manually activated.

7. Container according to claim 1 wherein the means for disinfection comprises one or more ultraviolet light sources, where the lights are activated either automatically or manually when the rack is inside the housing.

8. Container according to claim 1 wherein the housing comprises mounting means on the outside of the wall of the housing, where said means are adapted to engage a surface, such as a table top, kitchen sink or the like.

9. Container according to claim 1 wherein a separate push button device is provided, either separately from the container or integrated in the rack or closure member, where said push button device is connected to the disinfecting means and/or the means for moving the rack for activation of said means.

10. Sink, in particular a kitchen sink, where said sink comprises a basin and an upper flange surrounding said basin, where a container unit for storage and disinfection of various items, such as dishcloths, sponges, brushes and the like, where the unit comprises an outer housing, and an inner moveable rack, characterised in that the inner moveable rack comprises a reservoir, and that said rack and reservoir are connected to the outer housing by means whereby the inner rack can move out from a position in the housing where the housing substantially encloses the rack and reservoir, to a position where the rack and at least part of the reservoir are accessible, and that means are provided either in the housing or the rack for disinfecting objects on the rack is integrated or mounted in said flange.
